(19) United States
(12) Patent Application Publication
(10) Pub. No.: US 2008/0227953 A1
(43) Pub. Date: Sep. 18, 2008

(54) c-Mpl LIGAND POLYPEPTIDE

(75) Inventors: Lawrence A. Solberg, Jacksonville, FL (US); Frederic J. de Sauvage, South San Francisco, CA (US); Daniel L. Eaton, South San Fransisco, CA (US)

Correspondence Address: Schwegman, Lundberg & Woessner, P.A., P.O. Box 2938, Minneapolis, MN, 55402, US

(73) Assignee: Mayo Foundation for Medical Education and Research

(21) Appl. No.: 11/566,376

(22) Filed: Dec. 4, 2006

Related U.S. Application Data
(63) Continuation of application No. 08/362,662, filed on Dec. 22, 1994, now abandoned.

(51) Int. Cl. C07K 14/435

(52) U.S. Cl. 530/324

(57) ABSTRACT

A meg-CSF/thrombopoietin-like protein that is present in plasma of irradiated pigs has been purified. This protein, the porcine Mpl ligand polypeptide (ML), binds to and activates the c-Mpl receptor protein, a member of the cytokine receptor superfamily. The isolated Mpl ligand stimulates both megakaryocytopoiesis and thrombopoiesis.

This application is a continuation of U.S. application Ser. No. 08/362,662 filed on Dec. 22, 1994 which is incorporated herein by reference.

The present invention was made with the support of the National Institutes of Health under grant number HL17430. The U.S. Government has certain rights in the invention.

### BACKGROUND OF THE INVENTION

Platelet production has long been thought to be regulated by lineage-specific humoral factors. The plasma, serum and urine of thrombocytopenic animals and humans are thought to contain megakaryocytopoietic and thrombopoietic activities that are lineage specific and distinct from known cytokines. These activities, thought to be required for platelet synthesis, are referred to as meg-CSF or thrombopoietin on the basis of their ability to affect either proliferation (meg-CSF) or maturation (thrombopoietin) of megakaryocytes (N. Williams et al., J. Cell Physiol., 110, 101-104 (1982); N. Williams et al., Blood Cells, 15, 123-133 (1989)). Although these activities have been described since the 1960s, attempts to purify them have been unsuccessful.

Recently, the orphan cytokine receptor encoded by the c-mpl gene, known as c-Mpl receptor, has been implicated in the regulation of megakaryocytopoiesis. The expression of the c-mpl gene appears to be restricted to primitive stem cells, megakaryocytes and platelets, indicating its downregulation during the differentiation of all hematopoietic lineages except megakaryocytes. Moreover, c-mpl antisense oligonucleotides selectively inhibit megakaryocytic colony formation in vitro without affecting the growth of erythroid and granulomacrophage colonies (N. Methia et al., Blood, 82, 1395-1401 (1993)). These results suggest that a putative ligand for the c-Mpl receptor may be a megakaryocyte lineage-specific growth factor protein related to the factor(s) responsible for the meg-CSF or thrombopoietin activities present in thrombocytopenic plasma. Isolation and purification of this polypeptide ligand would in turn enable the evaluation of its role in regulating megakaryocytopoiesis and thrombopoiesis, as well as its potential to influence other hematopoietic lineages, and its therapeutic potential.

### SUMMARY OF THE INVENTION

The invention provides an isolated and purified polypeptide which binds to the c-Mpl receptor, which polypeptide is termed the Mpl ligand polypeptide (ML). Preferably, the Mpl ligand polypeptide of the invention is a polypeptide isolated and purified from aplastic porcine plasma, but a polypeptide within the scope of the term “Mpl ligand polypeptide” may also be obtained from the blood, plasma, serum, tissue, or other biological material obtained from any species, including human. Preferably, the N-terminal amino acid sequence of the Mpl ligand polypeptide is at least 70% identical with the N-terminal amino-acid sequence N-Ser-Pro-Ala-Pro-Pro-Ala-Cys-Asp-Pro-Arg-Leu-Leu-Asn-Lys-Leu-Leu-Arg-Asp-Asp-His-Val-Leu-His-Gly-Arg-Leu (SEQ ID NO:1), more preferably at least 80%, and most preferably is 100% identical with said sequence. The Mpl ligand polypeptide of the invention can bind to an affinity chromatographic column comprising an ML binding domain of an Mpl receptor. The Mpl ligand polypeptide of the invention can stimulate proliferation of a murine interleukin-3-dependent pro-B-cell line construct expressing a Mpl receptor (BA/F3-mpl) construct cultured in the absence of interleukin-3 (IL-3). Subunits of the polypeptide ligand preferably elute on a 4-20% SDS-PAGE gel at positions corresponding to Mᵣ values of about 30 kD, 28 kD and 18 kD.

Polypeptides within the scope of the invention can be used, in vitro or in vivo, to stimulate mammalian megakaryocytopoiesis. For example, intravenous administration of an appropriate Mpl ligand polypeptide can increase platelet levels in subjects in need of such treatment, or can be used as a stimulatory factor in the in vitro culture of hematopoietic stem cells.

### BRIEF DESCRIPTION OF THE FIGURES

FIG. 1 shows the results of two different assays for ML activity: (a) stimulation by aplastic porcine plasma (APP), and inhibition by Mpl receptor-IgG, of human in vitro megakaryocytopoiesis using the liquid suspension megakaryocytopoiesis assay, and (b) Mpl-receptor-dependent cell proliferation utilizing the Ba/F3-mpl cell proliferation assay.

FIG. 2 shows identification of porcine ML by (a) SDS-PAGE (silver stain) and (b) gel elution followed by activity assay utilizing the Ba/F3-mpl cell proliferation assay.

FIG. 3 shows strategies for detecting and cloning ML from other species.

### DETAILED DESCRIPTION OF THE INVENTION

The invention provides a polypeptide, referred to as Mpl ligand polypeptide (ML), which binds to the orphan cytokine receptor c-Mpl receptor. The ML can be the porcine protein or polypeptide, although Mpl ligand polypeptides of other species are also within the scope of the invention, and can be obtained as described below. Bioactive subunits or fragments of the native polypeptide are also within the scope of the invention, and can be obtained as described below.

As used herein, c-mpl is the gene encoding a protein known as the c-Mpl receptor. The c-Mpl receptor is also known as simply the Mpl receptor. The c-mpl gene (“c” referring to cellular) is the protooncogene and is distinct from but related to the v-mpl gene (“v” referring to viral), the latter being a cellular oncogene expressed by the myeloproliferative leukemia virus (MPLV) (I. Vigon et al., Proc. Natl. Acad. Sci. USA, 89, 5640-5644 (1992)).

The Mpl receptor is a membrane spanning receptor protein comprising extracellular, membrane spanning, and intracellular domains. The Mpl ligand (ML), also referred to as the Mpl ligand polypeptide, is a polypeptide ligand that binds to the extracellular portion of the Mpl receptor, thereby causing a signal to be transduced through the receptor.

Two forms of the human Mpl receptor have been cloned (I. Vigon et al., Proc. Natl. Acad. Sci. USA, 89, 5640-5644 (1992)). These human Mpl receptors differ only in their intracellular (cytoplasmic) domains. MPLK is the Mpl receptor encoded by the “K” clone MPLK, isolated by Vigon et al., which has an intracellular domain of 66 amino acids. MPLP is the Mpl receptor encoded by the “P” clone MPLP, isolated by Vigon et al., which receptor has an intracellular domain of 122 amino acids. The human receptor proteins MPLK and MPLP are reported by Vigon et al. to have identical extracellular and transmembrane domains.

The porcine Mpl ligand polypeptide of the invention can be obtained from aplastic porcine plasma (APP). Typically, pigs are rendered aplastic by irradiation. Platelet-poor plasma is collected by removing total blood volume, heparinization, and centrifugation. ML is isolated and purified from the resultant aplastic porcine plasma (APP) using a series of chromatographic steps, preferably hydrophobic interaction chromatography followed by immobilized dye chromatography followed in turn by affinity chromatography utilizing bound Mpl receptor. Typically, APP is first subjected to hydrophobic chromatography, preferably in the form of a phenyl-Toyopearl column. The active fractions are then exposed to an immobilized dye, preferably a blue-Sepharose column, and eluted with a buffer containing urea. Mpl receptor affinity chromatography, preferably utilizing tandemly linked CD4 receptor-IgG and Mpl receptor-IgG Ultralink (Pierce) columns, may then be used to further purify the ML. All or a portion of an Mpl receptor protein may be used to make the Mpl-receptor affinity column, provided that the portion used contains an ML binding domain. The Mpl receptor protein used in the Mpl-receptor affinity column is preferably isolated and purified from a recombinant cell line expressing a cloned human c-mpl gene (I. Vigon et al., Proc. Natl. Acad. Sci. USA, 89, 5640-5644 (1992); V. Mignotte et al., abstract, Blood, 80, 245a (1992)). ML elution from the Mpl-receptor affinity column is facilitated by the use of a low pH elution buffer.

The presence of ML may be monitored during the purification process by following a biological activity, as by using either the liquid suspension megakaryocytopoiesis assay or the Ba/F3-mpl cell proliferation assay, described below, most preferably by using the Ba/F3-mpl cell proliferation assay. Samples may be analyzed for the presence of ML by subjecting aliquots to sodium dodecyl sulfate-polyacrylamide gel electrophoresis (SDS-PAGE), eluting the protein bands from the gel, and assaying for biological activity as described. ML activity has been found to be associated with porcine protein bands which migrate at positions corresponding to Mᵣ values of about 30, 28 and 18 kD.

A novel Mpl-receptor-dependent cell proliferation assay, known as the Ba/F3-mpl cell proliferation assay, is provided by the invention. This assay utilizes a mammalian cell line expressing Mpl receptor to detect the presence of ML in a biological sample. This assay utilizes a human c-mpl gene stably expressed in a mammalian cell line, such as a primate cell line or the murine interleukin-3-dependent pro-B-cell line Ba/F3, (R. Palacios et al., Cell, 41, 727-734 (1985)). The addition of APP or other source of ML stimulates proliferation of the recombinant Ba/F3-mpl cells cultured in the absence of interleukin-3 (IL-3). This biological activity is completely abolished by the addition of soluble Mpl receptor-IgG fusion protein (described below).

To create a cell line for the Ba/F3-mpl cell proliferation assay, a DNA fragment corresponding to the entire coding sequence of human mpl P, the gene from the “P” clone MPLP encoding the human MPLP receptor, (I. M. Vigon et al., Proc. Natl. Acad. Sci. U.S.A., 89, 5640-5644 (1992)) may be obtained by polymerase chain reaction (PCR) using template cDNA, for example, cDNA prepared from the human megakaryocytic CMK cell line, and cloned into an appropriate vector, for example, pRK5-tkneo. Alternatively, mpl K, the gene from the “K” clone MPLK encoding the human MPLK receptor, or other gene encoding an Mpl receptor, can be used in place of mpl P. After linearization, the mpl construct can be introduced into Ba/F3 cells by any convenient technique, preferably by electroporation. Individual clones expressing Mpl receptor can be identified by, for example, fluorescence activated cell sorting (FACS) analysis using rabbit polyclonal antiserum raised against an Mpl receptor-IgG fusion protein. Stimulation of proliferation of Ba/F3-mpl cells of the assay in response to aplastic pig plasma (APP) or other source of ML can be measured by the extent of ³H-thymidine incorporation in the DNA. Incorporated radioactivity can be determined by methodologies known to the art.

The present invention also utilizes a modified version of another assay, known as the liquid suspension megakaryocytopoiesis assay and described in B. Grant et al., Blood, 69, 1334-1339 (1987). This assay detects a biological activity of ML in the form of megakaryocyte stimulation and is useful in determining whether ML is present in APP or other biological samples. It too may be used as a method of monitoring the presence of ML during the isolation and purification process. The assay assesses the effect of a biological sample on human in vitro megakaryocytopoiesis. In vitro stimulation of megakaryocytopoiesis was found to be indicative of the presence of ML, since a human Mpl receptor-IgG fusion protein containing the extracellular domain of Mpl receptor neutralizes this activity.

The Mpl receptor-IgG fusion protein used in the two assays described above may be obtained through recombinant genetic engineering. The Mpl receptor portion of the fusion protein comprises the extracellular domain of Mpl receptor. A cDNA fragment encoding human Mpl receptor or portion thereof encoding the extracellular domain of the receptor may be obtained by PCR from a cDNA library, and fused in-frame to a cDNA encoding the Fc region of human IgGl, as described in B. D. Bennett et al., J. Biol. Chem., 266, 23060-23067 (1991). The Mpl receptor-IgG construct is subcloned into a convenient vector and transfected into an appropriate cell line, for example, human embryonic kidney 293 cells, using any commonly available method for transfection, preferably the calcium phosphate method, as described by C. Gorman in DNA Cloning: A New Approach, 2, 143-190, (IRL, Washington, 1985). Mpl receptor-IgG expression from isolated clones can be determined using a human Fc-specific enzyme-linked immunosorbent assay (ELISA), and the recombinant Mpl receptor-IgG fusion protein can be purified using standard laboratory techniques for antibody purification, preferably using a protein A-Sepharose column (Pharmacia).

N-terminal amino acid sequence analysis of porcine ML isolated and purified from APP as described above yields the following unique N-terminal amino acid sequence: N-Ser-Pro-Ala-Pro-Pro-Ala-Cys-Asp-Pro-Arg-Leu-Leu-Asn-Lys-Leu-Leu-Arg-Asp-Asp-His-Val-Leu-His-Gly-Arg-Leu (SEQ ID NO:1), wherein “N-” signifies the N-terminus of the polypeptide and the other letters represent the standard one-letter amino acid abbreviations used in the art. The invention thus further provides a biologically active Mpl polypeptide ligand such that the N-terminal amino acid sequence is at least about 70% identical with the 26 residue N-terminal amino acid sequence N-Ser-Pro-Ala-Pro-Pro-Ala-Cys-Asp-Pro-Arg-Leu-Leu-Asn-Lys-Leu-Leu-Arg-Asp-Asp-His-Val-Leu-His-Gly-Arg-Leu (SEQ ID NO:1), where percentage identity is defined as the number of identical amino acids between an N-terminal sequence of the ML and SEQ ID NO:1, after such sequences have been aligned to maximize the number of identical amino acids, divided by 26. Preferably, the percentage identity between the N-terminal sequence of the ML and SEQ ID NO:1 is at least about 80%. More preferably, the N-terminal sequence of the ML is N-Ser-Pro-Ala-Pro-Pro-Ala-Cys-Asp-Pro-Arg-Leu-Leu-Asn-Lys-Leu-Leu-Arg-Asp-Asp-His-Val-Leu-His-Gly-Arg-Leu (SEQ ID NO:1). The term “biologically active” as used herein is defined as a biological activity which is at least 90% of the biological activity of the most active porcine Mpl ligand polypeptide, as determined by the assays described in the working examples.

Knowledge of the N-terminal sequence for porcine ML makes it possible to detect and isolate ML from other species using standard methods of molecular biology and recombinant DNA technology, some examples of which are described below. Accordingly, the present invention also provides an Mpl ligand polypeptide (ML) isolated and purified from other species. Preferably, the invention provides a human ML.

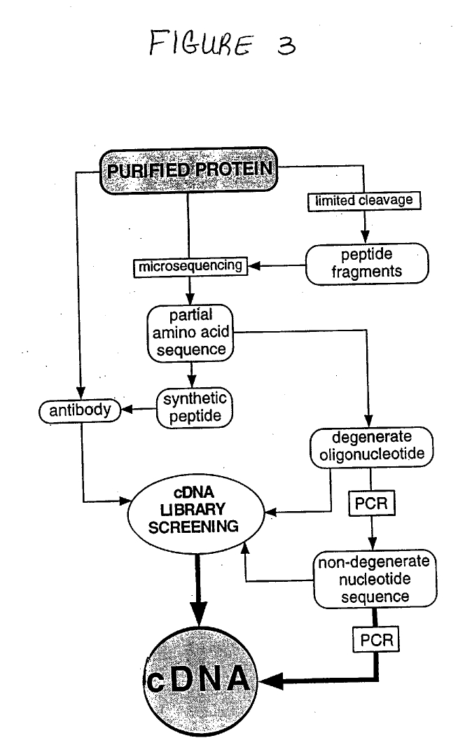

Several methods are available for using the N-terminal sequence of a protein from one species to detect the gene for a homologous protein in a different species and to isolate the cDNA clones of interest. Commonly used approaches are shown schematically in FIG. 3. Typically, a partial amino acid sequence is used to design degenerate oligonucleotide probes or primers for use in the generation of unique, nondegenerate nucleotide sequences by (PCR). These nondegenerate nucleotide sequences can in turn be used to design probes that can be used to screen cDNA libraries for the homologous gene of interest. For example, degenerate oligonucleotide primer pools designed on the basis of the N-terminal sequence obtained for the purified porcine ML can be used to amplify porcine genomic DNA by PCR. The sequence of the resulting amplification product, if it is encoded by a single exon, can in turn be used to synthesize a nondegenerate oligonucleotide that can be used to screen a genomic DNA library from a different species, preferably a human genomic library.

Positive ML clones from other species can be further characterized, subcloned and expressed using recombinant DNA methods well-known in the art. In eukaryotes, a source of messenger RNA may be needed for cDNA cloning. Reverse-transcribed PCR (RT-PCR) analysis can be performed on mRNA prepared from appropriate cell types, preferably human fetal and adult tissues and cell lines. This technique uses as primers oligonucleotides corresponding to the ends of an exon sequence identified from a positive cDNA clone.

As an alternative to the use of oligonucleotide primers and probes for the detection of related proteins in other species, antibodies raised against purified proteins can be used to identify positive clones from cDNA expression libraries.

### EXAMPLE I

### Stimulation by APP, and Inhibition by Mpl Receptor-IgG, of Human in vitro Megakaryocytopoiesis Using the Liquid Suspension Megakaryocytopoiesis Assay

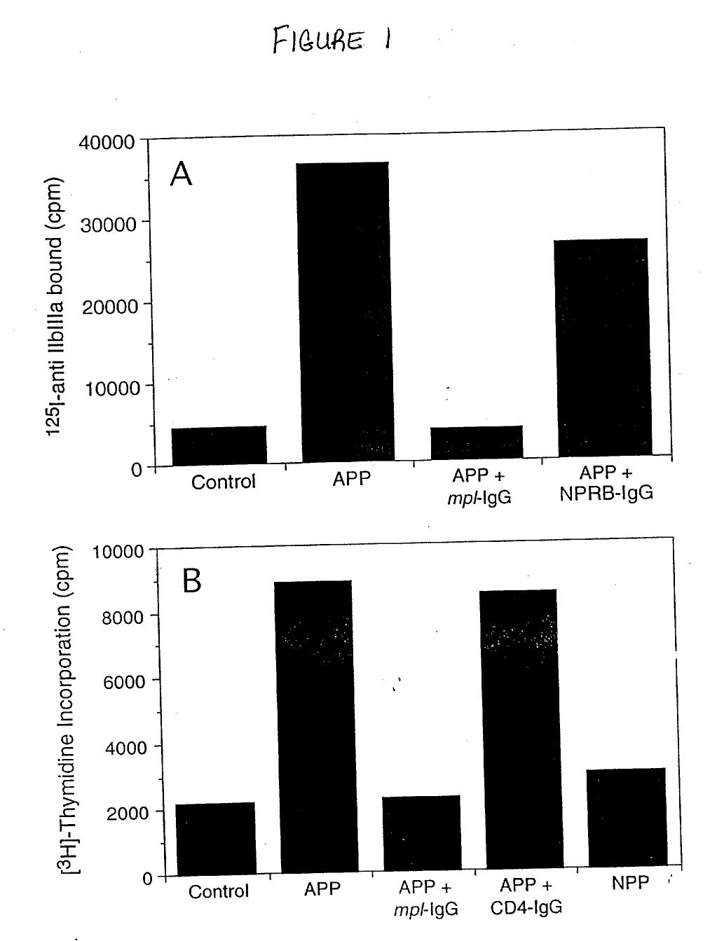

APP obtained from irradiated pigs was found to stimulate human megakaryocytopoiesis in vitro (FIG. 1(a)). To determine whether this stimulation was dependent on the presence of ML, a human Mpl receptor-IgG fusion protein containing the extracellular domain of Mpl receptor (Example II) was used in an attempt to neutralize this activity.

Platelet-poor plasma was collected from normal or aplastic anaemic pigs. Pigs were rendered aplastic by irradiation with 900 cGy of total body irradiation using a 4 meV linear accelerator. The irradiated pigs were supported for 6-8 days with intramuscular injections of cefazolin. Subsequently, their total blood volume was removed under general anaesthesia, heparinized, and centrifuged at 1,800×g for 30 min to make platelet-poor plasma. The megakaryocyte-stimulating activity was found to peak 6 days after irradiation.

The effect of APP on human megakaryocytopoiesis was determined using a modification of the liquid suspension megakaryocytopoiesis assay described by B. Grant et al., Blood, 69, 1334-1339 (1987), incorporated herein by reference. Circulating peripheral blood progenitor cells (PSC) obtained from consenting patients were diluted fivefold with phosphate buffered saline (PBS) and centrifuged at 800×g for 15 min at room temperature. The cell pellets were resuspended in Iscove's modified Dulbecco's media (IMDM) and layered onto 60% percoll (density 1.077 g-ml⁻¹, Pharmacia) and centrifuged at 800×g for 20 min. The light-density mononuclear cells were collected at the interface and washed twice with IMDM and plated out at 1-2×10⁶ cells per ml in IMDM containing 30% FBS (fetal bovine serum) (1 ml final volume) in 24-well tissue culture clusters (Costar). The cultures were incubated with or without 10% APP for 12-14 days in a humidified incubator at 37° C. in 5% CO₂ and air; 0.5 μg ml⁻¹ Mpl receptor-IgG (obtained as described in Example 11) or natriuretic peptide receptor/IgG (NPRB-IgG) (B. D. Bennett et al., J. Biol. Chem., 266, 23060-23067 (1991))) was added at days 0, 2 and 4. After 12-14 days in culture, megakaryocytopoiesis was quantified using a radiolabelled murine IgG monoclonal antibody (HP1-1D) against the megakaryocyte-specific glycoprotein GPIIbIIIa (provided by W. L. Nichols, Mayo Clinic) as described in B. Grant et al., Blood, 69, 1334-1339 (1987). The average of duplicate determinations is shown in FIG. 1(a). There was less than 10% variation between each duplicate.

It was found that Mpl receptor-IgG inhibited the megakaryocytopoietic activity of APP, whereas a control (natriuretic peptide receptor-IgG) fusion protein had no effect (FIG. 1(a)). This indicates that APP contains an ML, and that this ligand is necessary for the property of this plasma to stimulate megakaryocytopoiesis in vitro.

### EXAMPLE II

### Production of Mpl Receptor-IgG Fusion Protein

A cDNA fragment encoding amino acids 1-491 of human Mpl receptor (which region contains the extracellular domain of human Mpl receptor) was obtained by PCR from a human megakaryocytic CMK cell cDNA library and fused in-frame to a cDNA encoding the Fc region of human IgGl, as described in B. D. Bennett et al., J. Biol. Chem., 266, 23060-23067 (1991), incorporated herein by reference. The Mpl receptor-IgG construct was subcloned into pRK5-tkneo and transfected into 293 human embryonic kidney cells by the calcium phosphate method (C. Gorman in DNA Cloning: A New Approach, 2, 143-190, (IRL, Washington, 1985)). The cells were selected in 0.4 mg ml⁻¹ G418 and individual clones were isolated. Mpl receptor-IgG expression from isolated clones was determined using a human Fc-specific ELISA. The recombinant Mpl receptor-IgG was purified on a protein A-Sepharose column (Pharmacia).

### EXAMPLE III

### Development of the Ba/F3-mpl Cell Proliferation Assay

To facilitate purification of ML, an Mpl-receptor-dependent cell proliferation assay was developed. It was known that the stable expression of several different growth factor receptors in factor-dependent cell lines confers responsiveness to the respective growth factor (R. Palacios et al., Cell, 41, 727-734 (1985); A. D. D'Anrea et al., Mol. Cell. Biol., 11, 1980-1987 (1991); T. Kitamura et al., Proc. Natl. Acad. Sci. USA, 88, 5082-5086 (1991)). It was also known that the cytoplasmic domain of c-Mpl receptor can transduce a proliferation signal (R. C. Skoda et al., EMBO J., 12, 2645-2653 (1993); I. Vigon et al., Oncogene, 8, 2607-2615 (1993)) and that the extracellular domain of c-Mpl receptor appeared to bind ML (Example I, FIG. 1(a)). Speculating that the c-Mpl receptor might be therefore a single-chain receptor, the c-mpl gene was accordingly expressed in the murine interleukin-3-dependent cell line, Ba/F3 (R. Palacios et al., Cell, 41, 727-734 (1985)) and the ability of APP to stimulate proliferation of the Ba/F3-mpl cells was examined.

Specifically, a DNA fragment corresponding to the entire coding sequence of human mpl P, the gene from the “P” clone MPLP encoding the human MPLP receptor (I. M. Vigon et al., Proc. Natl. Acad. Sci. USA, 89, 5640-5644 (1992)) was obtained by PCR using as template cDNA prepared from the human megakaryocytic CMK cell line and was cloned into pRK5-tkneo. After linearization, this construct was introduced in Ba/F3 cells by electroporation (250 volts, 960 μF). Neomycin-resistant cells were selected in 2 mg ml⁻¹ G418 and individual clones were obtained by limiting dilutions. Clones expressing MPLP receptor, a human Mpl receptor, were identified by FACS analysis using rabbit polyclonal antiserum raised against human Mpl receptor-IgG. Stimulation of proliferation of Ba/F3-mpl cells in response to aplastic pig plasma was measured by the extent of ³H-thymidine incorporation in the DNA. Cells were starved of IL-3 for 16 hours then seeded in 96-well plates at a density of 25,000 cells per well in media containing APP at various concentrations in the presence or absence of Mpl receptor-IgG or CD4 receptor-IgG (D. J. Capon et al., Nature, 337, 525-531 (1989)) at 200 pM. After incubation for 22 hours, 1 μCi ³H-thymidine per well was added and the cells were incubated for an additional 6 hours before being collected. Incorporated radioactivity was determined in the presence of 40 μl of scintillation fluid (μicroscint 20) using a Top Count Counter (Packard Instruments). The average of duplicate determinations is shown in FIG. 1(b). There was less than 10% variation between each data point. One unit of activity produced 50% maximal stimulation.

Stable expression of the human c-mpl P gene did indeed confer APP responsiveness to Ba/F3-mpl cells cultured in the absence of TL-3 (FIG. 1(b)). This activity was completely abolished by soluble Mpl receptor-IgG, but not by the control fusion protein (CD4 receptor-IgG). Normal porcine plasma did not stimulate the Ba/F3-mpl cells. These results indicate that APP contains a factor(s) that can bind the extracellular domain of Mpl receptor and transduce a proliferative signal.

### EXAMPLE IV

### Purification of Porcine Mpl Ligand Polypeptide from APP

Mpl ligand polypeptide (ML) was purified from APP using hydrophobic interaction, immobilized dye, and Mpl receptor affinity chromatography. Specifically, aplastic porcine plasma (APP) obtained from irradiated pigs was made 4 M in NaCl and stirred for 30 min at room temperature. The resultant precipitate was removed by centrifugation at 3,800 r.p.m. in a Sorvall RC3B and the supernatant was loaded onto a phenyl-Toyopearl column (220 ml) equilibrated in 10 mM sodium phosphate containing 4 M NaCl. The column was washed with this buffer until the absorbance at 280 nm was <0.05 and eluted with H₂O. The eluted protein peak was diluted with H₂O to a conductivity of 15 mS and loaded onto a blue-Sepharose column (240 ml) equilibrated in phosphate buffered saline (PBS).

Subsequently, this column was washed with 5 column volumes each of PBS and 10 mM sodium phosphate containing 2 M urea. The column was eluted with 10 mM sodium phosphate containing 2 M urea and 1 M NaCl. The eluted protein peak was made 0.01% in octyl glucoside (n-octyl β-D-glucopyranoside) and 1 mM each in EDTA and Pefabloc (Boehringer Mannheim) and loaded directly onto tandemly linked CD4 receptor-IgG (D. J. Capon et al., Nature, 337, 525-531 (1989)) and Mpl receptor-IgG Ultralink (Pierce) columns. The CD4 receptor-IgG (2 ml) column was removed after the sample was loaded and the Mpl receptor-IgG (4 ml) column washed with 10 column volumes each of PBS and PBS containing 2 M NaCl, then eluted with 0.1 M glycine-HCl, pH 2.25. Fractions were collected into 0.1 volume of 1 M Tris, pH 8.0. The CD4 receptor-IgG and Mpl receptor-IgG columns were made by coupling 10-20 mg of each to 0.5 g of Ultralink (UL) resin according to the manufacturer's instructions.

A summary of the purification of the ML from 5 liters of APP is shown in Table 1.

TABLE 1
Purification of Mpl ligand polypeptide (ML) from APP

| | | | | | Specific | | |
|---|---|---|---|---|---|---|---|
| | | Protein | Units | | Activity | | x-Fold |
| Sample | Volume (ml) | (mg ml⁻¹) | ml⁻¹ | Units | (unit mg⁻¹) | Yield (%) | Purification |
| APP | 5,000 | 50 | 40 | 200,000 | 0.8 | — | 1 |
| Phenyl | 4,700 | 0.8 | 40 | 188,000 | 50 | 94 | 62 |
| Blue-S | 640 | 0.93 | 400 | 256,000 | 430 | 128 | 538 |
| Mpl-UL | 12 | 0.0005 | 1,666 | 20,000 | 3.3 × 10⁶ | 10 | 4.1 × 10⁶ |

ML activity present in column fractions was determined using the Ba/F3-mpl cell proliferation assay described in Example III. The overall purification from 5 liters of APP was greater than 4×10⁶-fold, with about 10% recovery of activity. The specific activity of the ligand eluted from the Mpl receptor affinity column was estimated to be ˜3×10⁶ units mg⁻¹.

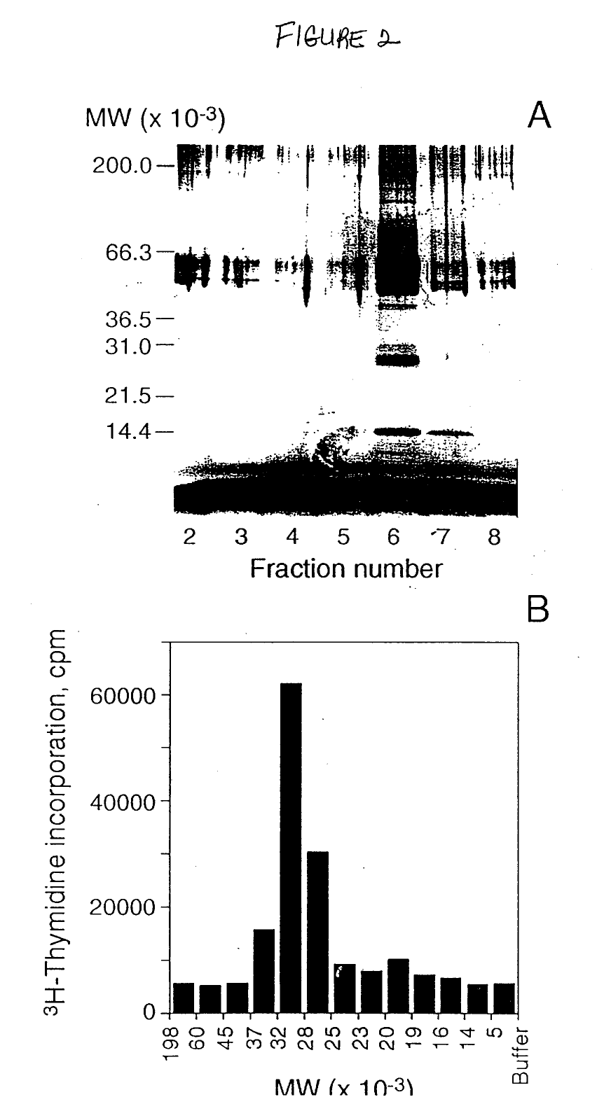

Aliquots (200 μl each) of the eluted fractions numbers 2 to 8 from the Mpl-receptor affinity column were precipitated with acetone and solubilized in Laemmli-SDS sample buffer. The samples were resolved on a 4-20% SDS-polyacrylamide gel (Novex) and proteins were visualized by silver staining (FIG. 2(a)). Several proteins were detected, the most intensely staining of which migrated at positions corresponding to M r values of ˜66K, 55K, 30K, 28K and 14K.

To determine the size of the ML, the affinity-purified ML (50 μl) was resolved on a Novex 14% SDS-polyacrylamide gel run under non-reducing conditions. The sample was not heated. As a control, sample buffer without ligand was applied to a separate gel under identical conditions. Following electrophoresis the gel was cut into 12 slices, noting their position in relation to M r markers. The 12 gel slices were placed into the cells in two Biorad model 422 electro-eluters and proteins were electro-eluted as previously described (M. G. Harrington, Meth. Enzymol., 182, 488-495 (1990)). The eluted proteins from the 12 gel slices were dialyzed overnight at 4° C. against PBS. Samples were then incubated on ice for 1 hour. Precipitated SDS was removed by centrifugation in a microfuge. The supernatants were then again placed on ice for 1 hour and microfuged. The final supernatants were diluted in PBS and assayed for Ba/F3-mpl cell proliferation activity (FIG. 2(b)).

Most of the activity was found in the 28 kD to 32 kD region of the gel, with a smaller amount of activity eluting in the 18 kD to 20 kD region. The only proteins visible in these regions had M r values of 30 kD, 28 kD and 18 kD.

To obtain sequence information on these proteins, the affinity-purified preparation was separated by SDS-PAGE and electroblotted onto polyvinylidenefluoride (PVDF) (P. Matsudaira, J. Biol. Chem., 262, 10035-10040 (1987)). N-terminal amino acid sequence analysis (S. C. Wong et al., Techniques in Protein Chemistry, (ed. Analetti, R. H.) 371-378 (Academic, San Diego, 1993)) of these three proteins gave the identical sequence, N-Ser-Pro-Ala-Pro-Pro-Ala-Cys-Asp-Pro-Arg-Leu-Leu-Asn-Lys-Leu-Leu-Arg-Asp-Asp-His-Val-Leu-His-Gly-Arg-Leu (SEQ ID NO:1), indicating that these proteins are derived from a common precursor. Computer-assisted analysis showed this amino acid sequence to be unique. Furthermore, this protein(s) is probably an ML because it coelutes with activity from an SDS gel at two different regions along the gel.

It will be appreciated by those skilled in the art that various modifications can be made to the above-described embodiments of the invention without departing from the essential nature thereof. The invention is intended to encompass all such modifications within the scope of the appended claims.

1. (canceled)

2. The polypeptide of claim 12 isolated and purified from aplastic porcine plasma.

3.-5. (canceled)

6. The polypeptide of claim 12 which binds to an affinity chromatographic column comprising an Mpl ligand polypeptide binding domain of an Mpl receptor.

7.-9. (canceled)

10. An isolated and purified Mpl ligand polypeptide comprising the N-terminal amino acid sequence set forth in SEQ ID NO:1, wherein said polypeptide stimulates human megakaryocytopoiesis in vitro and said polypeptide, or subunit thereof, migrates on a 4-20% gradient SDS-PAGE gel at positions corresponding to a Mr value selected from the group consisting of about 30 kD, 28 kD and 18 kD.


SEQUENCE LISTING (1 sequence)

SEQ ID NO: 1 - protein, 26 aa
SPAPPACDPRLLNKLLRDDHVLHGRL